(19) United States
(12) Patent Application Publication
(10) Pub. No.: US 2022/0062628 A1
(43) Pub. Date: Mar. 3, 2022

(54) METHOD OF USING AN ALVEOLAR BONE REGENERATION DEVICE

(71) Applicant: Medical Corporation Tominaga Dental Clinic, Naruto-shi (JP)

(72) Inventor: Toshihiko TOMINAGA, Naruto-shi (JP)

(73) Assignee: Medical Corporation Tominaga Dental Clinic, Naruto-shi (JP)

(21) Appl. No.: 17/134,458

(22) Filed: Dec. 27, 2020

(30) Foreign Application Priority Data
Aug. 31, 2020 (JP) 2020-145406

(51) Int. Cl. A61N 1/05; A61C 5/40; A61N 1/32

(52) U.S. Cl. CPC A61N 1/0548; A61N 1/326; A61C 5/40

(57) ABSTRACT

A method of using of an alveolar bone regeneration device including an active needle electrode covered totally or partially with insulation and an energization controller includes: inserting an electrode into the root canal of an alveolar bone and attaching a counter electrode to the mouth of a person; and performing energization between the electrode and the counter electrode using the energization controller. The energization is performed by one-shot energization using the current of a tone burst wave. Heat generated at a tip portion of the electrode sterilizes a tooth pulp, a root of a tooth, a periodontal tissue, and/or a lesion. The heat can kill bacteria at the tooth pulp, the root of the tooth, the periodontal tissue, and/or the lesion; activate osteoblasts, and regenerate an alveolar bone.

### TECHNICAL FIELD

The present invention relates to a method of using an alveolar bone regeneration device. More specifically, the present invention relates to a method of using an alveolar bone regeneration device acting as therapeutic equipment for regenerating, into a healthy state, an alveolar bone absorbed by bacteria.

### BACKGROUND ART

In the structure of a human tooth, the root of the tooth is surrounded and supported by the alveolar bone and is joined to the diaphysis of the jaw. Moreover, the gingiva surrounds the tooth at the top of the alveolar bone. If a human tooth is decayed and bacteria which have reached the dental nerves reach the vicinity of the root of the tooth through the root canal, pathogenic factors are released to the alveolar bone, so that the bone is melted and absorbed. This is called a periapical lesion.

If the alveolar bone is absorbed by the periapical lesion, the supporting force from the alveolar bone is lost, causing the tooth to become loose. When the alveolar bone is absorbed by bacteria at a level of up to two-thirds of the root of a tooth or 8 mm of the bone, the current treatment is tooth extraction.

However, once extracted, a tooth cannot be regenerated, and thus treatment without extraction is preferable.

Patent Literature 1 describes the related art for treating a periapical lesion.

As illustrated in FIG. 7, the related art is an apparatus (10) for treating and deactivating the vascular nerve bundle of a decayed tooth. The apparatus includes an electric circuit electrically connected to a first electrode 100 in the form of a needle to be inserted into an opening of a tooth, a second electrode (64) to be attached to the body of a patient, and a neutral gripping handle (60) electrically connected to the second electrode (64). The electric circuit includes an RF pulser unit that generates a high-frequency pulse for destroying or coagulating a vascular nerve bundle in contact with the first electrode (100). The second electrode (64) is attached to the lip of a patient, and the tip position of the first electrode (100), which is guided by a root canal connected to the root apex of a tooth, is measured. Following this, the high-frequency pulse is transmitted from the RF pulser unit to the first electrode (100).

However, the related art only deactivates the tooth pulp and cannot cure a periapical lesion.

### CITATION LIST

Patent Literature 1: Japanese Patent No. 4041165

### SUMMARY OF INVENTION

### Technical Problem

In view of the circumstances, an object of the present invention is to provide a method of using an alveolar bone regeneration device that can perform regenerative therapy on an alveolar bone by sterilizing a tooth pulp, a root of a tooth, a periodontal tissue, and/or a lesion, and activating osteoblasts.

### Solution to Problem

A method of using an alveolar bone regeneration device according to a first invention, the alveolar bone regeneration device including an active needle electrode provided with an electrode covered totally or partially with insulation and an energization controller for energizing the active needle electrode; is characterized by: inserting the electrode of the active needle electrode into the root canal of an alveolar bone along with attaching a counter electrode to the corner of the mouth, and sterilizing a tooth pulp, a root of a tooth, a periodontal tissue, and/or a lesion with heat generated at the tip portion of the active needle electrode through energization between the active needle electrode and the counter electrode by the energization controller.

In the method of using an alveolar bone regeneration device according to a second invention, in the first invention, the tip portion of the active needle electrode is brought to a tooth pulp, a root of a tooth, a periodontal tissue, and/or a lesion, heat is generated at the tip portion of the active needle electrode, and sterilization is directly performed on the tooth pulp, the root of the tooth, the periodontal tissue, and/or the lesion by using the heat.

In the method of using an alveolar bone regeneration device according to a third invention, in the first invention, the narrowed root canal portion is sterilized by Joule heat generated at the narrowed root canal portion remote from the tip portion, with the electrode of the active needle electrode inserted into the root canal of the alveolar bone.

In the method of using an alveolar bone regeneration device according to a fourth invention, in the first invention, the energization controller performs one-shot energization by using a current of a tone burst wave.

### Advantageous Effects of Invention

According to the first invention, heat is generated at the tip portion of the active needle electrode, and this high-temperature and electrical energy kill bacteria at a tooth pulp, a root of a tooth, a periodontal tissue, and/or a lesion, thus activating osteoblasts. When bacteria are killed and osteoblasts are activated, a periapical lesion is healed, and an alveolar bone can be regenerated.

According to the second invention, the tip portion of the active needle electrode can be brought to a tooth pulp, a root of a tooth, a periodontal tissue, and/or a lesion, and heating treatment can be efficiently performed.

According to the third invention, even if the tip portion of the active needle electrode cannot reach the narrowed root canal portion, it can still reach the tooth pulp and the root of the tooth in the area concerned, and heating treatment can be performed on the tooth pulp and the root of the tooth.

According to the fourth invention, treatment is completed after one-shot energization is performed one to several times. After the energization treatment, the root canal is filled. This eliminates the need for outpatient visits over many days and only requires a few visits to complete the treatment.

### BRIEF DESCRIPTION OF DRAWINGS

FIG. 1 is an explanatory diagram of an alveolar bone regeneration device A according to the present invention;

FIGS. 2(A) and 2(B) are explanatory drawings of an active needle electrode B;

FIG. 3 is an explanatory diagram illustrating a method of using the active needle electrode B according to the present invention;

FIGS. 4(A) and 4(B) are explanatory diagrams illustrating a method of using the active needle electrode B according to the present invention;

FIGS. 5(A), 5(B), 5(C), and 5(D) are tables indicating the results of treatment using the alveolar bone regeneration device A according to the present invention;

FIG. 6 is CT images showing the result of treatment—using the alveolar bone regeneration device A according to the present invention; and

FIG. 7 is an explanatory diagram of conventional high frequency therapy equipment.

### DESCRIPTION OF EMBODIMENTS

An embodiment of the present invention will first be described in accordance with the accompanying drawings.

An alveolar bone regeneration device A will be first described below before explaining a method of using an alveolar device bone regeneration device according to the present invention.

### (Alveolar Bone Regeneration Device A)

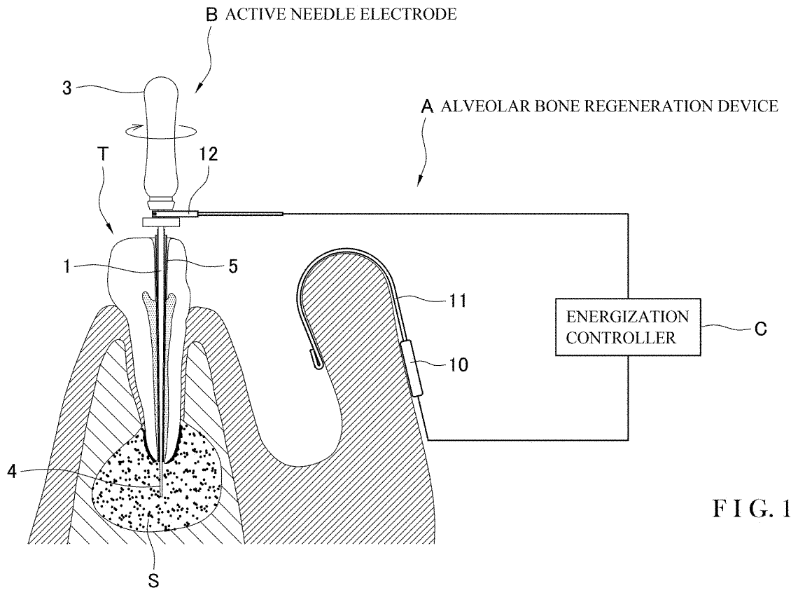

As illustrated in FIG. 1, the alveolar bone regeneration device A of the present invention includes an active needle electrode B and an energization controller C for energizing the active needle electrode B. In the alveolar bone regeneration device A, the active needle electrode B is inserted into a root canal and/or a lesion, the energization controller C applies power between the active needle electrode B and a counter electrode 10 attached to the corner of the mouth, and the alveolar bone regeneration device A is used for treatment. The members of the device will be described specifically below.

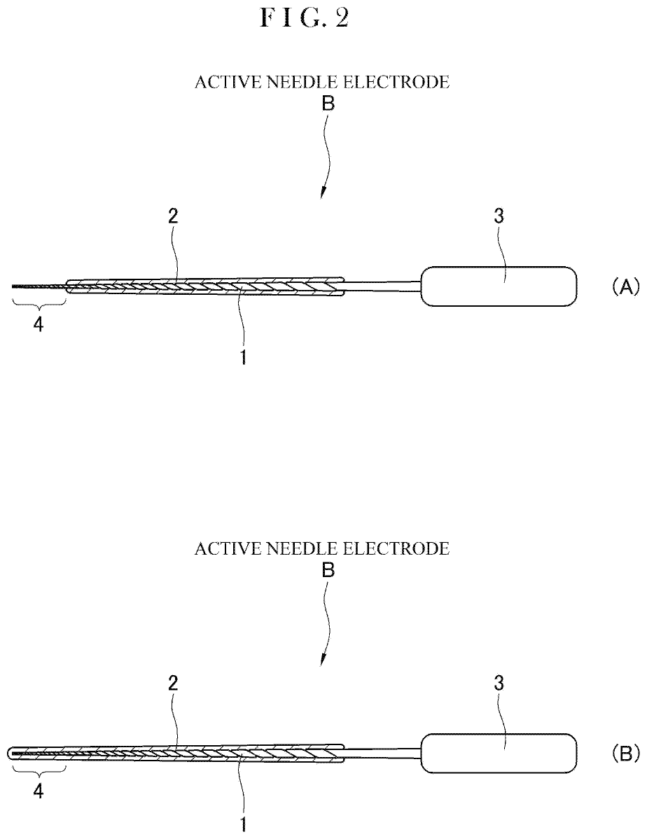

The active needle electrode B in FIG. 1 includes an electrode 1, a cover 2, and a handle part 3, as illustrated in FIGS. 2(A) and 2(B).

The handle part 3 is foamed at the proximal end of the electrode 1. The handle part 3 is made of a material that does not transfer heat to the hands, so that the handle part 3 is easily held by fingertips.

The electrode 1 is made of a metal that generates heat via energization and is free of corrosion, with high biocompatibility. Examples of such a metal include stainless steel and nickel titanium.

The electrode 1 is preferably 25.0 mm to 41.0 mm in length and is most preferably 31.0 mm in length. An electrode 1 having this length can insert a tip portion 4 deeply into the periapical lesion through a root canal. If the electrode 1 is shorter than 25.0 mm, the tip portion 4 cannot reach the periapical lesion and regenerative therapy cannot be performed on the tooth. On the contrary, if the electrode 1 is longer than 41.0 mm, problems such as bending or a breakage of the active needle electrode may occur, leading to difficulty in the handling of the electrode.

The electrode 1 is preferably 0.08 mm to 1.36 mm in diameter, and most preferably about 0.10 mm in diameter. An electrode 1 with such a diameter can smoothly pass through the root canal of the tooth, facilitating the heat generation necessary for treatment. If the diameter of the electrode 1 is smaller than 0.08 mm, the electrode 1 has lowered rigidity and is easily broken, leading to difficulty in the handling of the electrode. On the contrary, if the diameter is larger than 1.36 mm, it is difficult to pass the electrode 1 through the root canal, and a sufficient heat value cannot be obtained.

The electrode 1 may be shaped with parallels such that the proximal end and the tip have the same diameter, or the electrode 1 may taper down from the proximal portion toward the tip. The tip of the tapering electrode 1 is likely to bend and is easily inserted into the root canal of a tooth. The taper is made preferably at the ratio of 1/100 to 6/100 and particularly preferably at the ratio of about 2/100. The electrode 1 may be reversely tapered with a thin proximal portion and a thick tip.

The electrode 1 may have a needle shape that is circular, rectangular, or triangular in cross section from the proximal portion to the tip. Alternatively, the electrode 1 may be spiral in shape. A spiral electrode may have spiral grooves around a needle member or a spirally wound thin strip.

The spiral shape advantageously improves the contact of the cover 2, which will be described later, and prevents the cover 2 from accidentally falling off.

The cover 2 covers the outer circumference of the electrode 1 in order to limit the energized portion and generate heat.

The cover 2 is made of an insulating material, that is, an insulator. The insulator is not particularly limited and various materials may be used. However, a resin material is preferable in view of workability. The resin insulator may be any resin having an insulating function. Examples of the insulator include silicon resin, fluoroplastics (polytetrafluoroethylene, tetrafluoroethylene-hexafluoropropylene copolymer, tetrafluoroethylene-ethylene copolymer, tetrafluoroethylene-perfluoroalkyl vinyl ether copolymer), parylene resin, and polypropylene resin.

The portion that is energized and generates heat is a part of the electrode 1, and thus the cover 2 must not cover the entire surface 1 when the electrode 1 is used.

Thus, as illustrated in FIG. 2(A), the cover 2 is famed on a portion other than the part of the electrode 1 from the outset, or as illustrated in FIG. 2(B), the cover 2 is famed for the entire surface of the electrode 1 and is stripped off the part when the electrode 1 is used. Either one of the types may be used.

The energization controller C in FIG. 1 is a device connected to a direct-current or alternating-current power supply (e.g., a 100-V power supply for home use), so as to energize the alveolar bone regeneration device A. The energization controller C includes a known electric circuit. The energization controller C is used in combination with the counter electrode 10 having a large surface area relative to the active needle electrode B.

The active needle electrode B and the counter electrode 10 are connected to the energization controller C, the electrode 1 is inserted into a root canal, and a current is passed with the counter electrode 10 attached to the corner of the mouth, so that the current flows from the tip portion 4 of the electrode 1 to a periapical lesion S, a periodontal space, a gingiva, buccal mucosa, and the counter electrode 10. Thus, heat is generated at the tip portion 4 of the electrode 1 and transferred to portions to be treated, for example, a tooth pulp, a root of a tooth, a periodontal tissue, and/or a lesion.

During treatment, the heating temperature of the electrode 1 is preferably set at 40° C. to 120° C., and thus a current of 60-80 mA is preferably applied to the stainless electrode 1 having the foregoing dimensions. The application of the current can be implemented by a 100-V power supply for home use.

The counter electrode 10 in FIG. 1 may have any shape. The shape is not particularly limited and thus may be plate-like or rod-like.

At the root of the electrode 1, a clip 12 for fixing a lead wire is attached. The energization controller C is connected between the counter electrode 10 and the clip 12 via the lead wire.

In FIG. 1, the reference numeral 2 for the cover 2 provided for the electrode 1 is not indicated for simplicity of illustration. Reference numeral 5 denotes a guide sleeve for guiding the insertion of the electrode 1. The use of the guide sleeve 5 is optional.

### (Method of Use)

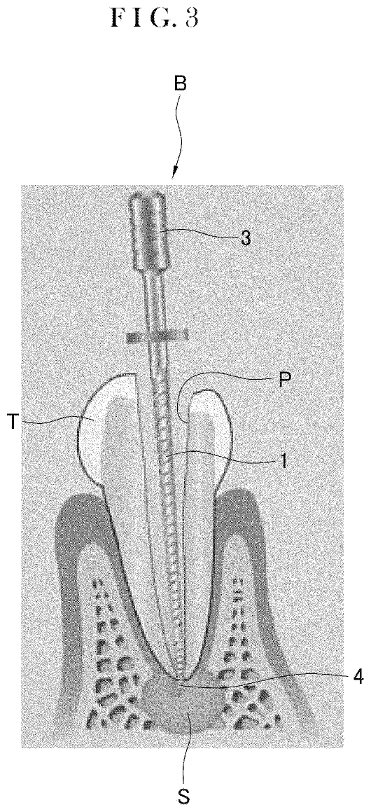

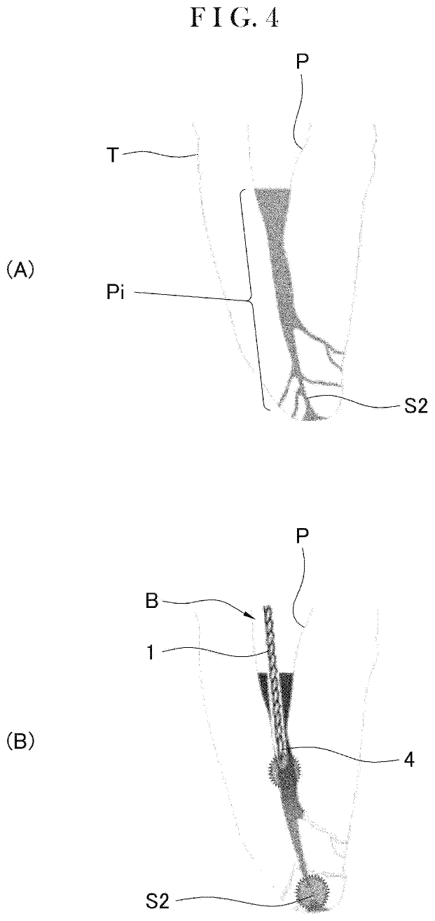

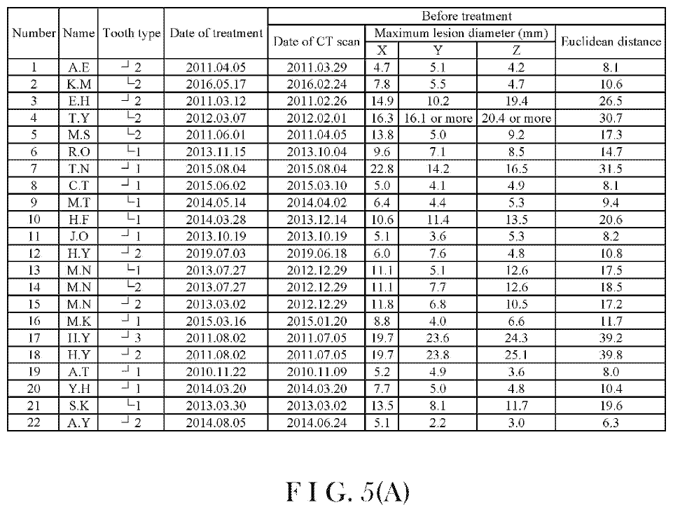

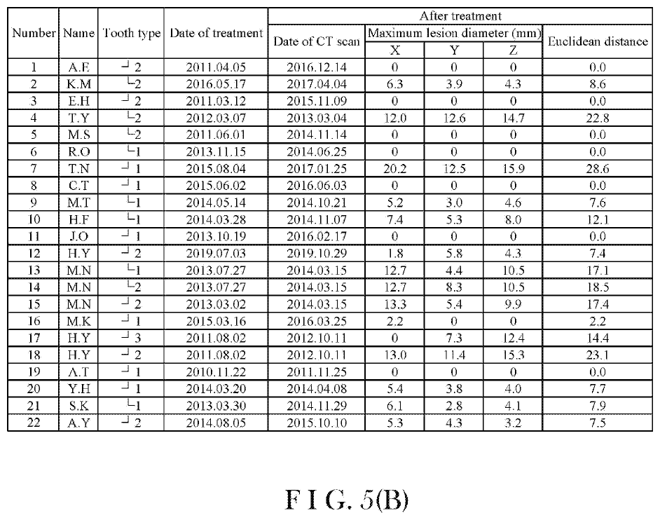

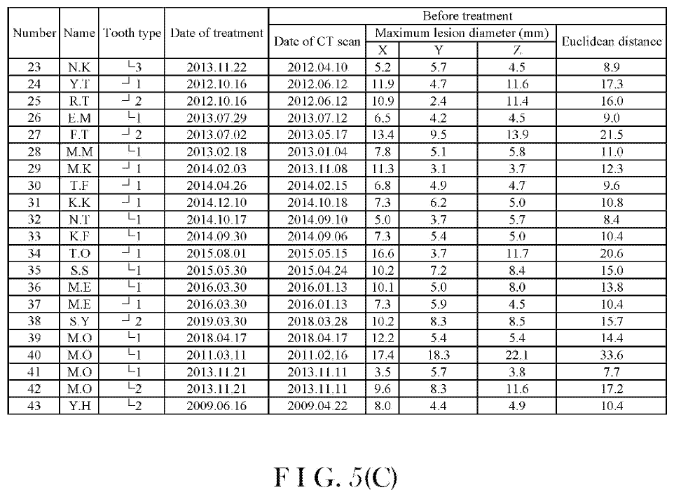

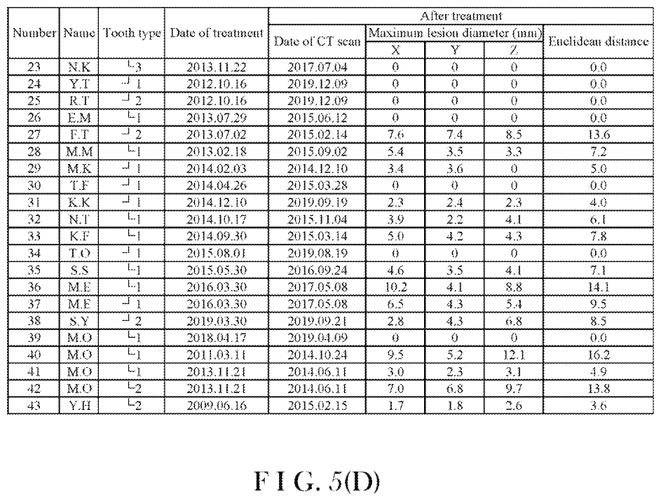

A method of using the alveolar bone regeneration device A according to the present invention will be described below with reference to FIGS. 1 and 3.

In FIG. 3, T denotes a tooth, P denotes a root canal, and S denotes a periapical lesion. As illustrated in FIG. 3, the electrode 1 of the active needle electrode B is inserted into the periapical lesion S through the root canal P of the tooth T. In other words, the tip of the electrode 1 has reached the periapical lesion S. As the electrode 1 is long and thin in shape, it can be brought to the periapical lesion S in this manner.

Even if the root canal P is curved, the electrode 1 is thin and easily bent, and thus can be deeply inserted into the root canal P. Even if the distance from the root canal P to the periapical lesion S is long, the electrode 1 itself having a sufficient length allows the tip of the electrode 1 therefore to reach the periapical lesion S.

### (First Method of Use)

As illustrated in FIG. 3, when heat is generated at the tip portion 4 of the electrode 1 having reached the lesion S, high-temperature and electrical energy can kill bacteria at a tooth pulp, the root of a tooth, a periodontal tissue, and/or a lesion, and activate osteoblasts. When bacteria are killed and osteoblasts are activated in this manner, the periapical lesion is healed, and the alveolar bone can be regenerated.

If the active needle electrode B in FIG. 2(B) is used in the above-described method of use, a portion of the cover 2 is stripped off from the electrode 1 such that the tip portion 4 is exposed by about 1.0 mm to 3.0 mm. Thus, electricity can be conducted and heat transmitted only to a tooth pulp, a root of a tooth, a periodontal tissue, and/or a lesion in that portion. If the active needle electrode B in FIG. 2(A) is used, the active needle electrode B can be used as it is.

Heat is typically generated from the tip portion 4 of the electrode 1. Thus, parts to be treated are changed by adjusting the insertion position of the electrode 1 to make it deeper or shallower. Furthermore, heating treatment can be performed on the different portions of a tooth pulp, a root of a tooth, a periodontal tissue, and/or a lesion, by gradually changing the insertion position of the tip portion 4.

“A tooth pulp, a root of a tooth, a periodontal tissue, and/or a lesion”, means any one, two, or three of a tooth pulp, a root of a tooth, a periodontal tissue, and a lesion; or all four of a tooth pulp, a root of a tooth, a periodontal tissue, and a lesion.

Since the heating portion is limited to the narrow scope mentioned above, any harmful effects to the living body are prevented, and the lesion can be treated.

### (Second Method of Use)

In FIG. 4(A), T denotes a tooth and P denotes a root canal. Pi in FIG. 4(A) denotes an infected tooth pulp. S denotes a narrowed root canal portion.

As illustrated in FIG. 4(B), the electrode 1 is inserted into the root canal P. Thereafter, the energization controller C passes a current between the electrode 1 and the counter electrode 10 so as to generate heat at the tip portion 4 of the electrode 1, whereas Joule heat is generated in the narrowed root canal portion S remote from the tip portion 4 of the electrode 1. In other words, collisions between electrons and positive ions are intensified in the narrowed root canal portion S, so that Joule heat is generated in the narrowed root canal portion S remote from the tip portion 4 of the electrode 1. By using the generated heat, heating treatment can be performed on a tooth pulp and a root of a tooth in the narrowed root canal portion S.

### (Method of Energization)

One-shot energization using a tone-burst wave is preferable in both the first method of use and the second method of use.

Regarding an energization waveform, it is more preferable that a tone-burst wave be used than a continuous wave. This is because the passage of the current of a continuous wave makes an incision in tissue, and the passage of a tone-burst wave with an interrupted waveform causes the thermocoagulation of tissue. A tone-burst wave in the present specification is an intermittent wave with a high crest factor, so that arc discharge is likely to occur, and discharged current is accumulated around a discharge point.

One-shot energization is energization in a short time, e.g., one second with, for example, a wattage of 20 W±10% and 10 pulses for 0.1 s. In one-shot energization, the temperature rises to around 40° C. to 120° C. at a tooth pulp, root of a tooth, a periodontal tissue, and/or a lesion, and sterilization is performed according to the temperature and electric energy. This activates osteoblasts, leading to bone regeneration.

The one-shot energization is performed on the lesion S (bone resorption portion) at intervals 1 mm to 2 mm. In other words, in the case of a lesion having a diameter of 18 mm, one-shot energization is successively performed on the portion at intervals of 1.0 mm to 2.0 mm, enabling six to seven shots. In the case of a lesion having a diameter of about 8 mm, one-shot energization is performed four times. If the lesion has a diameter of 2 mm, one-shot energization is performed a single time.

This treatment is completed after energization is performed one to several times. After the energization treatment, the root canal is filled. This eliminates the need for outpatient visits over many days and only requires a few visits to complete the treatment. The treatment for the lesion S can achieve the same treatment effects in a tooth pulp, a root of a tooth, and a periodontal tissue.

Treatment using the alveolar bone regeneration device A according to the present invention achieves early bone regeneration for the following two reasons.

### 1) Bactericidal Action

Bone resorption is suppressed due to the killing of bacteria at a tooth pulp, a root of a tooth, a periodontal tissue, and/or a lesion.

### 2) Activation of Osteoblasts

The differentiation and proliferation of osteoblasts which create bone is promoted.

### Examples

Bone regeneration effects based on a remedial example in which the alveolar bone regeneration device A according to the present invention was used will be described below.

### (Treatment Conditions)

Treatment using the alveolar bone regeneration device A according to the present invention was provided for patients having teeth with intractable periapical lesions whose clinical symptoms were unable to be brought into remission with conventional endodontic treatments, and teeth with considerable resorption of alveolar bones and an inability to bear occlusal force.

### (Results)

The tables in FIG. 5(A), FIG. 5(B), FIG. 5(C), and FIG. 5(D) indicate the treatment results.

The initials of the 43 subjects are indicated in the name fields of the tables. “Lesion” means a cavity created by osteolysis around the root of a tooth. Euclidean distances were calculated from the maximum diameters on the CT images and the changes in lesion sizes from before and after the operation were compared. In all cases, energization was performed under conditions of 500 kHz for one second per one time, and the number of shots was (maximum lesion diameter/2).

As indicated by the tables in FIG. 5(A), FIG. 5(B), FIG. 5(C), and FIG. 5(D), the lesions of 40 (93%) of the total 43 subjects were reduced in size, proving that the treatment was effective.

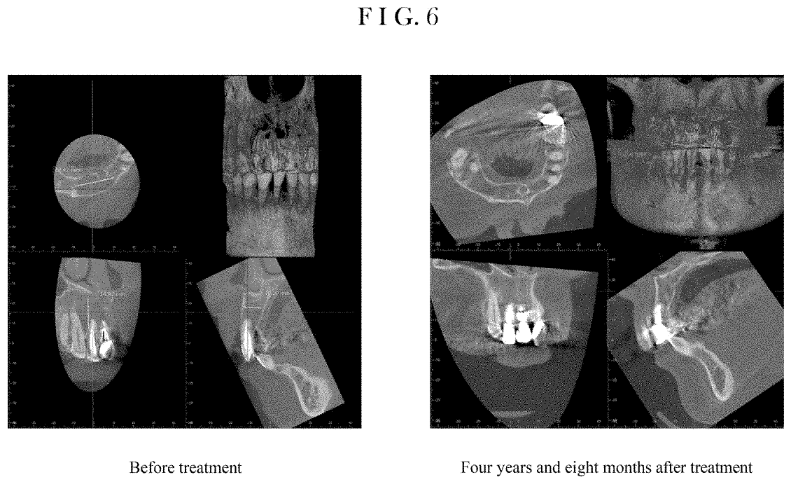

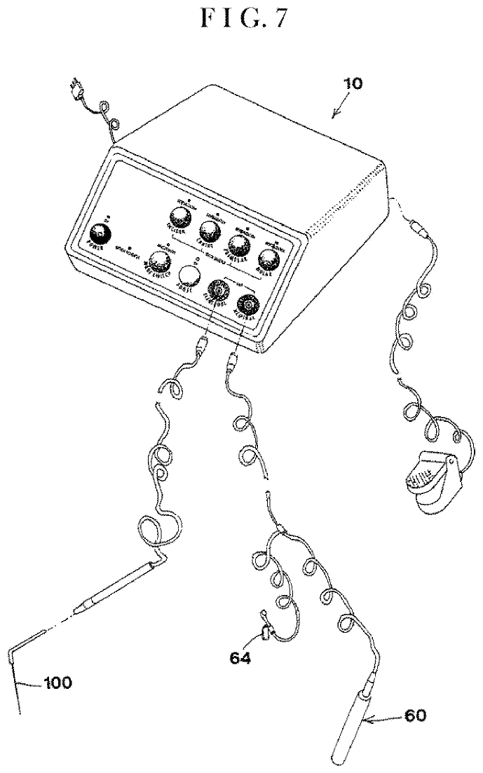

FIG. 6 shows CT images of the alveolar bones of a patient No. 3. The left CT image shows the state before the operation, and the left CT image shows the state four years and eight months after the operation. Lesions which were gray in the CT image before the operation are clear and white in the CT image after the operation. As is evident from the comparison, lesions having diameters of 14.9 mm×10.2 mm×19.4 mm were eliminated, proving that the alveolar bones were regenerated.

### REFERENCE SIGNS LIST

1 Electrode
2 Cover
3 Handle part
4 Tip portion
A Alveolar bone regeneration device
B Active needle electrode
C Energization controller

1. A method of using an alveolar bone regeneration device comprising an active needle electrode provided with an electrode covered totally or partially with insulation and an energization controller for energizing the active needle electrode,
the method comprising:
inserting the electrode of the active needle electrode into a root canal and attaching a counter electrode to the corner of the mouth; and
sterilizing a tooth pulp, a root of a tooth, a periodontal tissue, and/or a lesion with heat generated at a tip portion of the active needle electrode through energization between the active needle electrode and the counter electrode by the energization controller.

2. The method of using an alveolar bone regeneration device according to claim 1, wherein the tip portion of the active needle electrode is brought to a tooth pulp, a root of a tooth, a periodontal tissue, and/or a lesion to generate heat at the tip portion of the active needle electrode, and sterilization is directly performed on the tooth pulp, the root of the tooth, the periodontal tissue, and/or the lesion by using the heat.

3. The method of using an alveolar bone regeneration device according to claim 1, wherein the narrowed root canal portion is sterilized by Joule heat generated at the narrowed root canal portion remote from the tip portion, with the electrode of the active needle electrode inserted into the root canal of the alveolar bone.

4. The method of using an alveolar bone regeneration device according to claim 1, wherein the energization controller performs one-shot energization by using a current of a tone burst wave.
